(19) United States
(12) Patent Application Publication
(10) Pub. No.: US 2022/0359045 A1
(43) Pub. Date: Nov. 10, 2022

(54) PREDICTION OF ENZYMATICALLY CATALYZED CHEMICAL REACTIONS

(71) Applicant: INTERNATIONAL BUSINESS MACHINES CORPORATION, ARMONK, NY (US)

(72) Inventors: Matteo Manica, Zurich (CH); Teodoro Laino, Rüschlikon (CH); Daniel Probst, Aarau (CH); Alain Claude Vaucher, Zurich (CH)

(21) Appl. No.: 17/302,591

(22) Filed: May 7, 2021

(51) Int. Cl. G16C 20/70; G16C 20/30; G16C 20/10; G16C 20/90; G06F 16/33; G06F 16/338

(52) U.S. Cl. CPC G16C 20/70; G16C 20/30; G16C 20/10; G16C 20/90; G06F 16/3334; G06F 16/338

(57) ABSTRACT

Disclosed is a method for predicting at least one aspect of an enzymatically catalyzed chemical reaction. The method comprises providing a trained machine learning model, and inputting one or two input strings into the training model. Each input string is selected from a group of strings consisting of: a string representation of at least one educts of the chemical reaction, a string representation of at least one product of the chemical reaction, and/or a string representation of amino acids of an enzyme which is supposed to transform the educts into the products in the reaction. The trained machine learning model predicts at least the one or more strings which were not provided as input and the prediction is performed as a function of the one or two strings provided as input. The method outputting the prediction result for predicting or optimizing the chemical reaction.

### BACKGROUND

The present invention relates to computational chemistry and more specifically to the computational prediction of aspects of an enzymatically catalyzed chemical reaction.

US-20160024689-A1 describes systems and methods for identifying enzymes for catalyzing biochemical reactions. The method includes receiving input of reaction(s) and/or target molecule(s) along with data associated with chemical conversion, determining functional and linker region(s) in the input, scanning a transformation library for the determined functional region(s) of the reaction(s) and/or the target molecule(s) to find similar functional region(s) within the transformation library, assigning the reaction(s) and/or target molecule(s) to group(s) of the transformation library showing a high similarity to the transformation, computing a metabolite similarity score of the reaction(s) and/or target molecule(s) with respect to one or more reactions of the assigned group, and identifying enzyme(s) associated with the reaction(s) of the assigned group having a high metabolite similarity score. A transformation library is also generated.

US-20170235923-A1 describes a method and a device for selecting a pathway for a target compound by combining biochemical and chemical processes together, wherein an input of at least one pathway for synthesis of a target compound or degradation into a target compound is received, hybrid arrangements of one or more reaction steps included in the at least one pathway are predicted, a pathway feasibility score is computed, and at least one hybrid arrangement is selected based on the pathway feasibility score.

US-20170121852-A1 describes a method and device for multi-directionally predicting a plurality of output molecules through reaction prediction steps, computing similarity between the multi-directionally predicted output molecules, and using the generated data to predict chemical pathways.

Djoumbou-Feunang et al. (BioTransformer: a comprehensive computational tool for small molecule metabolism prediction and metabolite identification. J Cheminform 11, 2 (2019)) describe BioTransformer, a software package for in silico metabolism prediction and compound identification, wherein BioTransformer combines a machine learning approach with a knowledge-based approach to predict small molecule metabolism in human tissues (e.g., liver tissue), the human gut as well as the environment (soil and water microbiota), via its metabolism prediction tool.

### SUMMARY

The invention provides for a computer-implemented method, a computer program product and a computer system as specified in the independent claims. Embodiments are given in the dependent claims.

In one aspect of the invention a computer-implemented method for predicting at least one aspect of an enzymatically catalyzed chemical reaction of interest is provided. The method comprises providing at least one trained machine learning model, wherein the model was trained to correlate a string representation of one or more educts, a string representation of one or more products and a string representation of amino acids of an enzyme which transforms the educts into the products. The method further comprises inputting one or two input strings into the trained model. Each input string is selected from a group of strings consisting of: a string representation of one or more educts of the chemical reaction of interest, a string representation of one or more products of the chemical reaction of interest and a string representation of amino acids of an enzyme which is supposed to transform the educts into the products in the reaction of interest. The method further comprises predicting by the at least one trained model the one or more strings of the group of strings which were not provided as input. The prediction is performed as a function of the one or two strings provided as input. The method further comprises outputting the prediction result for predicting or optimizing the chemical reaction of interest.

In another aspect the invention relates to a computer program product for predicting at least one aspect of an enzymatically catalyzed chemical reaction of interest. The computer program product comprises a computer-readable storage medium. The computer-readable storage medium has program instructions embodied therewith. The program instructions are executable by a processing circuit and cause the processing circuit to provide at least one trained machine learning model. The model was trained to correlate a string representation of one or more educts, a string representation of one or more products and string representation of amino acids of an enzyme which transforms the educts into the products. The program instructions further cause the processing circuit to input one or two input strings into the trained model. Each input string is selected from a group of strings consisting of a string representation of one or more educts of the chemical reaction of interest, a string representation of one or more products of the chemical reaction of interest and a string representation of amino acids of an enzyme which is supposed to transform the educts into the products in the reaction of interest. The program instructions further cause the processing circuit to predict by the at least one trained model the one or more strings of the groups of strings which were not provided as input. The prediction is performed as a function of the one or two strings provided as input. The program instructions further cause the processing circuit to output the prediction result for predicting or optimizing the chemical reaction of interest.

In another aspect the invention relates to a computer system for predicting at least one aspect of an enzymatically catalyzed chemical reaction of interest. The computer system comprises a processor and a computer-readable medium. The computer-readable medium comprises at least one trained machine learning model. The model was trained to correlate a string representation of one or more educts, a string representation of one or more products and a string representation of amino acids of an enzyme which transforms the educts into the products. The computer-readable medium further comprises computer-readable program code that causes the processor to input one or two input strings into the trained model, each input string being selected from a group of strings consisting of a string representation of one or more educts of the chemical reaction of interest, a string representation of one or more products of the chemical reaction of interest and a string representation of amino acids of an enzyme which is supposed the transform the educts into the products in the reaction of interest. The computer-readable program code further causes the processor to predict by the at least one trained model one or more strings of the groups of strings which were not provided as input. The prediction is performed as a function of the one or two strings provided as input. The computer-readable program code further causes the processor to output the prediction result for predicting or optimizing the chemical reaction of interest.

In another aspect the invention relates to a further computer-implemented method for training at least one untrained machine-learning model. The method comprises providing training data. The training data comprises string representations of one or more educts of chemical reactions, string representations of one or more products of chemical reactions, and string representations of amino acids of enzymes that are known to transform the educts into the products. The computer-implemented method further comprises inputting the training data into the at least one untrained machine learning model. The computer-implemented method further comprises training the at least one untrained machine learning model using the training data for providing at least one trained machine learning model. The at least one trained machine learning model is trained to correlate string representations of one or more educts, string representations of one or more products and string representations of amino acids of an enzyme which transforms the educts into the products.

In yet another aspect the invention relates to a computer program product for training at least one untrained machine-learning model. The computer program product comprises a computer-readable storage medium having program instructions embodied therewith. The program instructions are executable by a processing unit and cause the processing unit to provide training data. The training data comprises string representations of one or more educts of chemical reactions, string representations of one or more products of chemical reactions, and string representations of amino acids of enzymes that are supposed to transform the educts into the products. The program instructions further cause the processing unit to input the training data into an untrained machine learning model. The program instructions further cause the processing circuit to train the untrained machine learning model using the training data provided. The program instructions further cause the processing circuit to provide at least one trained machine learning model. The at least one machine learning model is trained to correlate string representations of one or more educts, string representations of one or more products and string representations of amino acids of an enzyme which transforms the educts into the products.

In yet another aspect the invention relates to a computer system comprising a processor and a computer-readable medium. The computer-readable medium comprises training data. The training data comprise string representations of one or more educts of chemical reactions, string representations of one or more products of the chemical reactions and string representations of amino acids of enzymes that are known to transform the educts into the products. The computer-readable medium further comprises computer-readable program code that causes the processor to input the training data into an untrained machine learning model. The computer-readable program code further causes the processor to train the untrained machine learning model using the training data whereby the machine learning model is trained to correlate string representations of one or more educts, string representations of one or more products and string representations of amino acids of an enzyme which transforms the educts into the products.

As will be appreciated by one skilled in the art, aspects of the present invention may be embodied as an apparatus, a method or a computer program product. Accordingly, aspects of the present invention may take the form of an entirely hardware embodiment, an entirely software embodiment (including firmware, resident software, microcode etc.) or an embodiment combining software and hardware aspects that may all generally be referred to herein as a circuit, module or system. Furthermore, aspects of the present invention may take the form of a computer program product embodied in one or more computer-readable mediums having computer executable code embodied thereon.

### BRIEF DESCRIPTION OF THE DRAWINGS

In the following, various embodiments of the invention will be described, by way of example only, and with reference to the drawings in which:

FIG. 1 shows a flowchart which illustrates a method for predicting the products of a chemical reaction of interest.

FIG. 2 shows a flowchart which illustrates a method for predicting the educts and enzyme of a chemical reaction of interest.

FIG. 3 shows a flowchart which illustrates a method for predicting the optimal amino acid sequence of an enzyme.

FIG. 4 shows a flowchart which illustrates a method for training an untrained machine learning model using training data.

FIG. 5 illustrates an example of a computer system that can be used for the prediction of various aspects of an enzymatically catalyzed chemical reaction of interest.

FIG. 6 shows a flowchart which illustrates a method for training a natural language processing model and applying the trained model to predict various aspects of enzymatically catalyzed chemical reactions of interest.

FIG. 7 illustrates examples of product prediction models, precursors-prediction models and enzyme-prediction models.

FIG. 8 shows a flowchart which illustrates a method for generating a trained predictive model by training the model on a training data set and predicting the products of a chemical reaction of interest.

FIG. 9 shows a flowchart which illustrates a method for generating a trained predictive model by training the model on a training data set and predicting the precursors of a chemical reaction (i.e., educts and enzymes).

FIG. 10 shows a flowchart which illustrates a method for generating a trained predictive model by training the model on a training data set and predicting the optimal amino acid sequence of an enzyme.

### DETAILED DESCRIPTION

The descriptions of the various embodiments of the present invention will be presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein the term ‘chemical reaction of interest’ refers to a chemical reaction which uses an enzyme to transform one or more educts into one or more products for which one or more elements are not yet known and/or are suspected to be not optimal. For example, the chemical reaction of interest may be a reaction where some or all of the educts may not be known, or where one or more of the products may not be known, or where the enzyme may not be known or where the amino acid sequence of the enzyme is suspected to be sub-optimal (in terms of an optimization criterion, e.g., efficiency, purity, speed, minimization of toxic substances etc.). So, a chemical reaction of interest is a chemical reaction which shall be characterized completely while at the moment the knowledge of the chemical reaction may be incomplete.

As used herein the term ‘educt’ or ‘reagent’ refers to a substance or compound consumed in the course of a chemical reaction.

As used herein the term ‘product’ refers to a substance or compound produced in the course of a chemical reaction.

A ‘catalyst’ as used herein is a substance or compound adapted to induce or increase the rate of a chemical reaction, whereby the catalyst is not consumed in the catalyzed reaction but can typically act repeatedly. ‘Catalyzed’ as used herein refers to a chemical reaction whose rate is increased by using a catalyst.

An ‘enzyme’ as used herein is a biochemical compound (e.g., a protein) that acts as a catalyst in a chemical reaction. The enzyme increases the rate of a chemical reaction and is not changed by the reaction. Enzymes are often constructed by a sequence of amino acids.

A ‘machine learning model’ or ‘model’ as used herein is an executable program and/or a set of parameters which is adapted to predict a particular outcome, e.g., to compute one or more output strings given one or more input strings. A model can use one or more classifiers in trying to determine the probability of a particular string or string element (symbol) to occur based on the provided input. Often, machine-learning models are referred to as ‘predictive models. The machine learning model might be, for example, a deep learning model, an artificial intelligence model, a natural language processing model or any other model know to a person skilled in the art.

An ‘unsupervised machine learning model’ as used herein is a type of machine learning algorithm that learns patterns from untagged or unlabeled data.

A ‘supervised machine learning model’ as used herein is a type of machine learning algorithm that learns patterns from tagged or labeled data.

A ‘semi-supervised machine learning model’ as used herein is a type of machine learning algorithm that combines a small amount of tagged or labeled data with a large amount of untagged or unlabeled data during training.

The term ‘green chemistry’, also called sustainable chemistry, is an area of chemistry and chemical engineering that focuses on the design of products and processes that minimize or eliminate the use and generation of hazardous substances. Green chemistry focuses on the environmental impact of chemistry, including reducing consumption of nonrenewable resources and technological approaches for preventing pollution. The goals of or the “criteria to be achieved by” green chemistry comprise more resource-efficient and inherently safer design of molecules, materials, products, and processes.

A ‘line notation’ as used herein is a typographical notation system using characters that is used for chemical nomenclature. The ‘line notation’ may be a notation which uses a sequence of string elements, typically symbols or symbol groups, to encode the structure and molecular composition of a substance.

‘Natural language processing’ as used herein is a sub-field of computer science and artificial intelligence concerned with the interactions between computers and human language, in particular how to program computers to process and analyze large amounts of natural language data.

A ‘natural language processing model’ as used herein a computational algorithm that can be used to process and analyze large amounts of natural language data.

‘Machine translation’ as used herein is automated translation or “translation carried out by a computer”, as defined in the Oxford English dictionary. Machine translation involves a translation model that may be selected from a group consisting of the following: a machine learning model, wherein the machine learning model may be supervised, semi-supervised, or non-supervised, a natural language processing model, a deep learning model, a sequence-to-sequence language processing model, and a transformer model.

A ‘Sequence-to-sequence language processing model (Seq2Seq model)’ as used herein is a natural language processing model that has been trained to convert sequences from one domain (e.g., sentences in English, SMILES sequences of educts) to sequences in another domain (e.g., the same sentences translated to French, SMILES sequences of products).

A ‘transformer model’ as used herein is a natural language processing model that has a so-called transformer architecture. The transformer architecture can also be referred to as an encoder-decoder architecture, wherein the encoder consists of a set of encoding layers that processes the input iteratively one layer after another and the decoder consists of a set of decoding layers that processes the output of the encoder iteratively one layer after another

A string as used herein is a sequence of characters (or ‘symbols’), e.g., as a literal constant or as some kind of variable.

The simplified molecular input line entry system (SMILES) is a specification in the form of a line notation for describing the structure of chemical species using short ASCII strings. SMILES strings can be imported by most molecule editors for conversion back into two-dimensional drawings or three-dimensional models of the molecules.

The term ‘SMILES arbitrarily targets specification’ (SMARTS) is a notation for specifying sub-structural patterns in molecules. The SMARTS line notation is expressive and allows extremely precise and transparent sub-structure specification and atom typing. SMARTS is related to the SMILES line notation that is used to encode molecular structures and, like SMILES, was originally developed by David Weininger and colleagues at the Daylight Chemical Information Systems.

The term ‘tokenization’ as used herein is the process of separating a piece of text into smaller units called tokens. Tokens can be either words, characters or sub-words.

The term ‘Deep learning’ as used herein relates to a machine learning approach which is based on artificial neural networks with representation learning. Learning can be supervised, semi-supervised or unsupervised.

Embodiments of the invention may be advantageous because they may provide a data-driven learning system for complete automatic planning of biochemical reactions.

Another advantage may be that the method can be used for complete automatic design of enzymes. This is advantageous because computer-driven, in-silico experiments are less expensive than wet lab research.

Another advantage may be that all elements of the reaction, including the enzyme, are represented as strings. This may allow predicting an optimized string representation which may not exist or may not be known yet. Hence, embodiments of the invention do not rely on and are not limited to predicting educts, products and/or enzymes known to exist and comprised e.g., in a chemical substance library, but may rather be able to predict and identify new reaction pathways, new substances and/or enzymes which are not yet known or examined. Embodiments of the invention do not rely on a library and are not limited to identifying one or more substances contained in the library. Rather, the string-based representation of all elements of the chemical reaction may allow predicting an (optimal) element at the level of individual symbols constituting the strings. The string-representation of the amino acid sequence may allow optimizing the amino acid sequence and/or correlating an amino acid sequence of an enzyme with educts which can be transformed by this amino acid sequence and/or with products whose creation can be catalyzed by this amino acid sequence.

Computationally predicting chemical reactions may allow avoiding the performing of cost-expensive experiments in the wet lab. For example, the method can be used to identify chemical reactions that can be used to synthesize a product of interest.

In one aspect, the invention relates to a computer-implemented method for predicting at least one aspect of an enzymatically catalyzed chemical reaction of interest and to a corresponding computer system and computer program product. The method comprises providing at least one trained machine learning model, wherein the model was trained to correlate a string representation of one or more educts, a string representation of one or more products and a string representation of amino acids of an enzyme which transforms the educts into the products. The method further comprises inputting one or two input strings into the trained model. Each input string is selected from a group of strings consisting of a string representation of one or more educts of the chemical reaction of interest, a string representation of one or more products of the chemical reaction of interest and a string representation of amino acids of an enzyme which is supposed to transform the educts into the products in the reaction of interest. The method further comprises predicting by the at least one trained model the one or more strings of the group of strings which were not provided as input. The prediction is performed as a function of the one or two strings provided as input. The method further comprises outputting the prediction result for predicting or optimizing the chemical reaction of interest.

For example, the method can be used to plan various synthesis steps.

According to some examples, the amino acid representation can be in the form of SMILES or SMARTS.

According to some further examples, the computer-implemented method further comprises generating the trained predictive model by training the model on a training dataset. The training dataset comprises a plurality of known enzymatically catalyzed chemical reactions, each reaction in the training datasets specifying one or more educts, one or more products and the enzyme which transforms the educts into the products. The molecular composition and structure of the educts, the products and the enzyme are specified in a string representation. The representation of the molecular composition and the structure of the educts, the products and the enzyme using a string representation might be advantageous because string representations can be easily processed in machine learning models such as natural language processing models.

Examples of the prediction method comprise processing textual representations of molecules (e.g., SMILES) leveraging the same or similar way as natural language processing techniques are used for processing natural language (e.g., for language translation). Especially, in the case of the product-prediction model and the precursor-prediction models the computational problem can then be solved by using a machine translation setting, where the machine learning algorithm learns how to translate the language (i.e. the textual representation) of educts and enzymes into the language (i.e. the textual representation) of the products and the language (i.e. the textual representation) of the products into the language (i.e. the textual representation) of educts and enzymes respectively. This machine translation problem can then be solved computationally using machine learning models with transformers architecture that provide an encoder and decoder.

For example, the computer-implemented method may use similar transformer models, and more in general, models acting on string representations of enzymes or their amino acid sequence and string representations of chemical reactions (e.g., reaction SMILES) to learn a direct mapping between the two aforementioned elements. These models can then be used to explore the enzyme space in various ways, for example, propose synthesis route to generate desired products, find amino acid sequences describing an enzyme that will optimize the reactions, find the optimal educt for a reaction, and ultimately perform de-novo design of enzymes.

According to some further examples, the computer-implemented method may be based on natural language processing models (e.g., a transformer model) that was pretrained on existing biochemical reaction datasets. The model takes string representations of enzymes/proteins (or their amino acid sequence) and educts (SMILES separated by specific characters, e.g., “.”) and predicts the product (SMILES) of the chemical reaction of interest. The same model can be trained, using the same data, to learn multiple mappings: product to educts and enzyme, educts and product to enzyme, educts and enzyme to products, product and enzyme to educts, educts to product and enzyme, or enzyme to educts and products. All these model instantiations can be used to explore synthesis routes in the biochemical space, allowing implicitly to account for the full enzyme sequence information. Once the model is pretrained it can be used for de novo enzyme design fixing the information of the educts and products of interest. The computer implemented method can, according to some examples, provide a user interface to select the different model types, hence allowing to discover novel enzymes and synthetic routes in a completely data driven fashion.

According to another example the training data may be derived from publicly available datasets or commercial data sets. Databases which may comprise data that can be used directly or after some processing as training data sets are, for example, the BRaunschweig ENzyme DAtabase (BRENDA), Rhea database, Pathbank, and/or MetaNetX.

According to some further examples, the machine learning models that are used as training models for the training data or as prediction models to predict the at least one step of a chemical reaction of interest further comprises data pre-processing steps. The data pre-processing steps may comprise the following or combinations thereof: tokenization, vectorization, sequence modelling (sequentialization). A “token” is a fundamental unit that is mapped to a learnable vector that is fed as sequence element into the transformer model. Starting from an input string the computer implemented method generates a list of tokens during the tokenization step. The list is then converted in a list vectors that are inputted into the machine learning models. The mapping between the vectors and the tokens is bijective and guarantees a one-to-one correspondence between a token and a vector. The tokenization can be performed in multiple ways. In the case of SMILES, for example, the tokenization is based on identifying atoms and using the characters or group of characters representing those as tokens. In the case of amino acid sequences, for example, the tokenization is based on not only on single amino acids, but also groups of amino acids (multiple characters) occurring with a high frequency in a database of protein sequences (e.g., one of the above-mentioned databases). In the case of enzyme commission (EC) number the tokenization is based on the classes and subclasses of the enzyme and the chemical properties that they are coding for. For example, tripeptide aminopeptidases have the code “EC 3.4.11.4”, whose components indicate the following groups of enzymes: “EC 3” enzymes are hydrolases (enzymes that use water to break up some other molecule), “EC 3.4” are hydrolases that act on peptide bonds, “EC 3.4.11” are those hydrolases that cleave off the amino-terminal amino acid from a polypeptide and “EC 3.4.11.4” are those that cleave off the amino-terminal end from a tripeptide. “EC 3.4.11.3” gets represented using four tokens “[v3]” “[u4]” “[t11]” “[q3]”.

In sequence modelling the machine learning algorithm does not only take the input parameters into the algorithm into account, but also models the sequential information that is provided by the order of the input parameters.

According to another example, the aspect to be predicted by the computer-implemented method is the one or more products that will be generated in the enzymatically catalyzed chemical reaction of interest. The at least one predictive model comprises a trained product prediction model which is used for performing the prediction. The product prediction model is adapted for predicting a string representation of one or more products of the chemical reaction of interest as a function of the string representation of one or more educts and of the amino acids of the enzyme. The one or more input strings which are input into the trained model are the string representation of the one or more educts of the chemical reaction of interest and the string representation of amino acids of the enzyme which is supposed to transform the educts into the products in the reaction of interest. The one or more strings predicted by the model is the string representation of the one or more products. This embodiment of the invention might be advantageous because it can be used for the synthesis of specific chemical products.

For example, the SMILES sequence of the educts is given as follows: “C(C(C(C(C(CO)O)O)O)O)O.O” and the abbreviated amino acid sequence is given as follows: “MPGQQATKHE . . . DGGYTTR”. The product prediction model then predicts the product as “C1C(C(C(C(O1)(CO)O)O)O)O”. Another advantage of the disclosed computer-implemented method may be that starting from a product of interest a completely automatic exploration of the biochemical space can be performed to find the best possible synthesis with the best possible enzyme. The data-driven learning system may be used for complete automated planning of biochemical reactions. This may be advantageous to explore more efficient synthesis routes for given pharmaceutical agents or narrowing down candidate pharmaceutical agents that have the potential to be synthesized given the biochemical space. The biochemical space may also be explored to identify target compounds that interact with the enzymes in a specific way so that they might represent novel targets for the synthesis of pharmaceutical agents.

Another advantage of the disclosed computer-implemented method may be that it may be used for predicting how a substance is going to be metabolized by certain enzymes that are found in various organs of the human body.

According to another example of the method, the computer-implemented method is used to predict the precursors required for producing the one or more products. The precursors comprise the educts and the enzyme of the chemical reaction of interest. The at least one predictive model comprises a trained precursors prediction model which is used for performing the prediction. The precursors prediction model is adapted for predicting a string representation of one or more educts and a string representation of the amino acid sequence of the enzyme of the chemical reaction of interest as a function of the string representation of one or more products. The one or two input strings which are inputted into the trained model is the string representation of the one or more products of the chemical reaction of interest. The one or more strings predicted by the model comprises the string representation of the one or more educts and the string representation of amino acids of the enzyme which is supposed to transform the educts into the products in the reaction of interest.

This may be advantageous because enzymes and educts are predicted that can be used to synthesize a specific product of interest.

According to some examples the method chemical reaction of interest represents a single step in a multistep synthesis plan. The computer-implemented method further comprises using the precursors prediction model recursively. The output of the previous execution of the precursor prediction model is then used as the input of subsequent executions of the precursor prediction model. The recursive usage of the model generates a multistep synthesis plan.

This may be advantageous as the method may be used to predict complex, multi-step synthesis plans, wherein the products of one reaction are used as educts in a subsequent reaction.

According to some examples of the method, another model, e.g., the product prediction model, and/or multiple models are used in each step. For example, a product prediction module may be used for predicting the products of a first chemical reaction and the product prediction model may be used in a second iteration to predict the products to be generated using the products of the first reaction as the educts of the second reaction. In addition, the enzyme-prediction module is used in each step for verifying if the amino acid sequence of the enzyme used or predicted in a given step can be optimized.

According to some example implementations, the synthesis plan is considered to be completed and the recursive use is automatically ended once a termination condition is met. The terminating condition is selected from a group consisting of: all predicted educts are commercially available, all predicted educts are non-toxic, all predicted educts are water soluble, all predicted educts meet a predefined requirement (e.g., in respect to purity, availability, price, storability), and a combination of two or more of the aforementioned conditions.

Said features may be advantageous because it can be used to automatically optimize single-step or multi-step chemical reaction plans in respect to one or more different criteria. For example, less quantities of solvents need to be used if only water-soluble reagents are used. Another advantage may be that only water-based reagents can be used in a chemical synthesis in order to avoid the use of organic solvents. Another advantage may be that the prediction automatically terminates once a reaction is predicted that involves only non-toxic educts and non-toxic products or that involves only educts which are commercially available. Another advantage of some examples of the method may be that the prediction may selectively predict and output reactions that have a high yield of the product of interest and only few side products. Another advantage of said features may be that the prediction method automatically terminates once a reaction is identified which meets green chemistry goals or requirements.

In another example of the computer-implemented method the aspect to be predicted is the optimal amino acid sequence of an enzyme capable of catalyzing the chemical reaction of interest. The at least one predictive model comprises a trained enzyme prediction model which is used for performing the prediction. The enzyme prediction model is adapted for predicting a string representation of the amino acid sequence of an enzyme optimally capable of catalyzing the chemical reaction of interest as a function of the string representation of the one or more educts and the one or more products of the chemical reaction of interest. The one or two input strings which are input into the training model are the string representation of the one or more educts and the string representation of the one or more products. The one or more strings predicted by the model is the string representation of amino acids of the enzyme which is supposed to be the optimum amino acid sequence for an enzyme capable of transforming the educts into the products in the reaction of interest.

Said features may have the advantage of allowing the discovery of novel enzymes and synthetic routes which is not limited to known enzymes comprised in a library: as the prediction is performed on a string representation of the amino acid sequence of the enzyme, the prediction may return an amino acid sequence which is predicted to provide optimum catalytic capabilities eve in case this enzyme does not exist or is not known to exist.

The method may be used to design new enzymes that are optimized to be used in specific synthesis routes without the use of expensive wet lab reactions. This may be advantageous to predict the optimal amino acid sequence of an enzyme that is specific for a certain reaction of interest.

According to another example the computer-implemented method further comprises checking the quality of the prediction of the optimal amino acid sequence. The computer-implemented method comprises inputting the string representation of the predicted optimum amino acid sequence and the string representation of the one or more educts of the reaction of interest into the at least one predictive model. The method further comprises, in response to the inputting, predicting by the trained model a string representation of one or more products to be generated by an enzyme having the predicted optimum amino acid sequence from the one or more educts based on the inputted string representations. The computer-implemented method further comprises determining if the said one or more predicted products are identical to the one or more products used as input for predicting the optimum amino acid sequence of the enzyme. If the products are identical the predicted optimum amino acid sequence is considered as verified. If the products are not identical the predicted optimum amino acid sequence is considered as non-verified and unreliable.

Said features may be advantageous as the above-mentioned determination step may allow automatically identifying the accuracy of a prediction without performing a wet lab step to verify the prediction. For example, the above-mentioned steps may be executed in order to computationally estimate the quality of the prediction and a final, wet-lab based verification may be performed only for reactions involving an enzyme whose optimum amino acid sequence was verified as described above.

According to some examples of the computer-implemented method, the at least one predictive model comprises three models trained on the same training data. The three models comprise a product prediction model adapted to predict a string representation of one or more products of the chemical reaction of interest as a function of the string representation of one or more educts and of the amino acids of the enzyme, a precursors prediction model adapted to predict a string representation of one or more educts and of the amino acids of the enzyme based on a string representation of one or more educts, an enzyme prediction model adapted to predict a string representation of the amino acid sequence of an enzyme optimally suited for catalyzing the transformation of one or more educts into one or more products based on a string representation of the educts and of the products.

Said features may be advantageous as the combination of two or more of the above-mentioned models may allow increasing the accuracy of the prediction, and verifying the predictions of other models. For example, if a product prediction model is used to predict the products given a set of educts and an enzyme having a particular amino acid sequence, an enzyme prediction model can be applied on the educts and the predicted products to predict the optimum amino acid sequence for the educts and the predicted products. If the predicted optimum amino acid returned in the second prediction step is identical to the amino acid sequence used as input in the first prediction, there is no need to optimize/amend the amino acid sequence of the enzyme. If the predicted optimum amino acid sequence differs from the actual amino acid sequence in the enzyme used in the first prediction, it may be advisable to synthesize an enzyme variant with the optimum amino acid sequence and to check whether this variant provides better reaction results.

Another embodiment of the invention relates to a computer-implemented method wherein the training data comprises additional information. The additional information is one or more of the following: toxicity information of at least some of the educts and/or products, efficiency information of the chemical reaction of interest, solubility information of at least some of the educts and/or products and/or the enzymes, selectivity information of the chemical reaction of interest.

The trained predictive model is configured to perform the prediction such that at least one of the following is optimized: the toxicity of the educts and/or products of the chemical reaction of interest is minimized, the quantities of necessary solvents is minimized, the need for organic solvents is minimized, the amount of unwanted side products is minimized, the yield of a target product is maximized.

This method may be advantageous because it can be used to reduce toxicity including environmental toxicity. Another advantage may be that this method can be used to predict chemical reactions or parts whereof which meet green chemistry requirements or goals. For example, efficient reactions can be planned, for example reactions which use less quantities of solvents. Another example would be that the models are trained to predict educts, products and/or enzymes of water-based reactions in order to avoid organic solvents. Another advantage may be that the selectivity or yield of the predicted reactions or reaction elements can be increased. For example, the models can be trained to predict and return reactions or reaction elements (e.g., educts, products or enzymes) of reactions that have a high yield of the target product and/or only generate few side products and/or only generate non-toxic products and/or only require non-toxic educts selectively or preferably.

Toxicity information might for example include environmental toxicity or substances that are toxic to the human body including carcinogenic substances. Solubility information might include information on water-based solubility, fat-based solubility of solubility in organic solvents. Selectivity information might include information on stereo-selectivity of reactions.

In a further aspect, a method of performing a chemical synthesis is provided. The method comprises performing the computer-implemented method described herein for embodiments and examples of the invention for predicting the totality of elements of a chemical reaction based on a subset of given elements. The elements comprise one or more educts, one or more products and an enzyme. The method further comprises combining the one or more educts and the enzyme of the chemical reaction of interest identified by the prediction and letting the enzyme transform the one or more educts into the one or more products. For example, this method might be done in a chemical or biological laboratory.

According to some examples, the method further comprises chemically or biologically synthesizing an enzyme having the predicted optimum amino acid sequence.

For example, the synthesis of an enzyme having the predicted optimized amino acid sequence can be performed in a cell culture. For example, the amino acid sequence of an existing enzyme can be modified by genetic engineering techniques such as CRISPR/CAS 9 in combination with cloning techniques for transferring a gene encoding the modified, optimized enzyme into an expression vector, e.g., a cell line, and for harvesting the enzyme from this cell line. For short enzymes, in particular peptide-based enzymes, a chemical synthesis may also be an option.

According to some examples, the computer-implemented method comprises creating a line notation being indicative of the structure and molecular composition of each of the educts using a sequence of string elements. Each string element is one of the following: a Unicode character (e.g., ASCII characters), an artificially created character representing an atom or atom group, a group of adjacent characters together representing an atom or atom group. The line notation is the simplified molecular input line entry system (SMILES), the Wiswesser line notation (WLN), the Representation of Organic Structures Description Arranged Linearly (ROSDAL), Sybyl line notation (SLN) or SMILES arbitrary target specification (SMARTS) or one letter or three letter amino acid sequence in case the educt or product is a peptide or a protein. If the one or more educts comprises more than one educt concatenating the line notation of each of the one or more educts and one or more delimiters to obtain a concatenate to be used as the line notation of the educts. The method further comprises using the line notation of the string representation of the one or more educts.

Said features may be advantageous as this notation allows a machine-learning model to learn to correlate individual atoms or atom groups (e.g., amino acids in the case of the enzymes) of different elements of a chemical reaction rather than to correlate whole molecules derived from a substance or enzyme library. As a consequence, the trained models may be able to predict the structure and composition of educts, products and/or enzyme on a very fine-grained manner and may be able to predict novel, unknown substances, enzymes and biochemical reactions.

According to some examples, the computer-implemented method further comprises creating the line notation of the one or more educts. Creating the line notation of the one or more educts comprises analyzing the molecular composition of a plurality of known molecules for identifying a predefined number of chemical groups occurring most frequently in the analyzed molecules. Each chemical group further comprises a plurality of atoms. Creating the line notation of the one or more educts further comprises representing each of the atoms of the educts not being a member of one of the identified chemical groups by a respective atom-specific symbol and identified chemical groups by a single symbol. Creating the line notation of the one or more educts further comprises representing each of the identified chemical groups occurring in the one or more educts by a respective chemical group-specific symbol, the chemical group-specific symbols being different from the atom-specific symbols.

Said features may have the advantage that the prediction speed may be significantly increased and the workload on the computational resources including the processing unit, for example the central processing unit (CPU) and/or the graphical processing unit (GPU) as well as the memory consumption may be significantly decreased. In addition, the method may be advantageous because the model training time is reduced and the model accuracy is increased. As the most frequently occurring atoms or atom groups in the reaction elements comprised in the training data are represented by a single symbol rather than a group or sequence of symbols, the string representation of the educts, products and/or of the amino acids of the enzyme is much shorter and hence the storing and processing of the strings consumes less computational resources and storage space. For example, the most frequently occurring groups of amino acids in the enzymes comprised in the training data set or the most frequently occurring sequence of functional groups comprised in educts in the training data may be represented by a single symbol. Instead of the training data, a different substance library whose content is similar to the trainings data may be used for determining the occurrence frequencies of atom groups. An atom group can be, for example, an amino acid or a functional group. As the occurrence frequencies of the atom groups determines if an atom group is represented by a single symbol or a sequence of symbols, the occurrence frequency dependent line representation may depend of the frequencies of atom groups in the evaluated substance data set, e.g., the training data set. Hence, the compression may implicitly be optimized for the substances in the chemical reaction data set used as training data. According to some examples, the computer-implemented method further comprises creating a line notation being indicative of the structure and molecular composition of each of the products using a sequence of string elements. Each string element is in particular one of the following: a Unicode character (e.g., ASCII characters), an artificially created character representing an atom or atom group, a group of adjacent characters together representing an atom or atom group. The line notation is in particular the simplified molecular input line entry system (SMILES), the Wiswesser line notation (WLN), the Representation of Organic Structures Description Arranged Linearly (ROSDAL), Sybyl line notation (SLN), or SMILES arbitrary target specification (SMARTS). If the one or more products comprise more than one product concatenating the line notations of each of the one or more products and one or more delimiters to obtain a concatenate to be used as the line notation of the products. The computer-implemented method further comprises using the line notation as the string representation of the one or more products.

Using the above-mentioned notations may have the advantage that many biological and chemical databases already comprise a string representation of their substances in one or more of the above-mentioned notations and hence can be used for training the models.

According to some examples, the creation of the line notation of the one or more products comprises: analyzing the molecular composition of a plurality of known molecules for identifying a predefined number of chemical groups occurring most frequently in the analyzed molecules. Each chemical group comprises a plurality of atoms. Creating the line notation of the one or more products further comprises representing each of the atoms of the products not being a member of one of the identified chemical groups by respective atom-specific symbol and identified chemical groups by a single symbol. Creating the line notation of the one or more products further comprises representing each of the identified chemical groups occurring in the one or more products by a respective chemical group-specific symbol, the chemical group-specific symbols being different from the atom-specific symbols.

As mentioned above, said features may reduce storage and consumption of processing resources (e.g., CPU/GPU usage, memory load) and may increase processing speed in particular during the training phase when the one or more models are created and a large amount of training data is processed, but also during the test phase when the trained model(s) are applied on input strings.

According to some examples, the computer-implemented method further comprises creating a line notation of amino acids of the enzyme wherein the line notation covers all amino acids or covers at least the amino acids of the enzymatically active moiety of the enzyme. The computer-implemented method further comprises using the line notation as the string representation of the enzyme.

This may be advantageous as it may allow optimizing the enzyme by applying a predictive model having learned to correlate individual amino acids or amino acid groups with the ability to transform one or more educts (and atom groups comprised therein) into one or more products (and atom groups comprised therein).

In particular, the line notation can be the one letter or three letter amino acid code sequence [which is specified e.g., in International Union of Pure and Applied Chemistry and International Union of Biochemistry: Nomenclature and Symbolism for Amino Acids and Peptides (Recommendations 1983). In: Pure & Appl. Chem. Band 56, Nr. 5, 1984, S. 595-624, doi:10.1351/pac198456050595].

According to some examples, the computer-implemented method further comprises creating the line notation of the amino acid sequence of the enzyme. Creating the line notation of the enzyme comprises analyzing the amino acid sequences of a plurality of known enzymes for identifying a predefined number of amino acid subsequences occurring most frequently in the analyzed enzymes. Each amino acid subsequence comprises two or more amino acids. Creating the line notation of the one or more products further comprises representing each of the amino acids of the enzyme not being a member of one of the identified subsequences by a respective amino acid-specific symbol, and representing each of the identified amino acid subsequences occurring in the enzyme by a respective subsequence-specific symbol, the subsequence-specific symbols being different from the amino acid-specific symbols.

For example, if the occurrence frequencies of a sequence of two or more amino acids in the training data exceeds a predefined threshold, the said sequence of amino acid is considered as a subsequence to be represented by a subsequence-specific symbol.

Said features may be advantageous as they may reduce the workload on computational resources, including bandwidth, storage consumption, usage of the processing unit (e.g., CPU and/or GPU) and memory consumption when storing and/or processing the string representations of the amino acid sequences of the enzymes, in particular during the training phase. In addition, the prediction speed may be significantly increased, the model training time may be reduced, and the model accuracy may be increased.

According to an embodiment of the present invention the computer-implemented method comprises at least one machine learning model. The at least one machine learning model is a natural language processing model adapted to translate string representing sequences in a source language into string representing sequences in a target language.

Applicant has surprisingly observed that some machine learning techniques which have originally been developed for a completely unrelated task such as interpreting and translating natural language text can successfully be used for predicting chemical reactions and for optimizing enzymes and synthesis pathways.

According to an embodiment of the present invention the natural language processing model is a non-supervised machine learning model.

In yet another embodiment of the present invention the machine learning model is a machine translation model that provides a translation algorithm to transform the input data into the output data. The machine learning model may be a natural language processing model that comprises a sequence-to-sequence language processing model or a transformer model.

The machine translation model may be of a different architecture as various architectures of machine translation models are known to the person skilled in the art. The machine translation model may be any model that can be used to model a sequence of data. When used iteratively or recursively as part of a multi-step prediction model machine translation models of various architectures may be used sequentially. For example, in a first step a sequence-to-sequence model may be used for product prediction, and in a second step a transformer model may be used for optimal enzyme prediction.

A ‘sequence-to-sequence natural language processing model’ as used herein is a machine learning model used for natural language processing that turns one sequence into another sequence by using an artificial neural network.

An ‘artificial neural network’ as used herein is a machine learning model that is capable of deep learning, i.e., learning unsupervised or semi-supervised from data that are unstructured and/or unlabeled. The artificial neural network may be selected from a group comprising recurrent neural networks, long short-term memory artificial neural networks, or gated recurrent unit artificial neural networks.

Recurrent neural networks are a class of artificial neural networks where connections between nodes form a directed graph along a temporal sequence.

A Long short-term memory artificial neural network is an artificial neural network with a recurrent neural network architecture that has feedback connections. An artificial neural network with gated recurrent units is a long short-term memory neural network with a forget gate. The sequence-to-sequence model comprises, for example, three parts: an encoder, an encoder vector, and a decoder. The encoder uses deep neural network layers and converts the input tokens (symbols or groups of symbols obtained by tokenizing the one or more string representation of substances provided as input to corresponding hidden vectors. Each vector represents the current token and the context of the token within its string representation. The decoder is similar to the encoder. It takes as input the hidden vector generated by encoder, its own hidden states and current word to produce the next hidden vector and finally predict the next token, whereby the predicted token represents an atom or atom group (e.g., an amino acid of an enzyme, a carboxyl group of a educt or product) of the predicted substance or enzyme.

A ‘transformer model’ as used herein is a machine learning model that uses deep learning models and is designed to handle sequential data and does not require that the sequential data be processed in order. The transformer has an encoder-decoder architecture, wherein the encoder consists of a set of encoding layers that processes the input iteratively one layer after another and the decoder consists of a set of decoding layers that does the same thing to the output of the encoder.

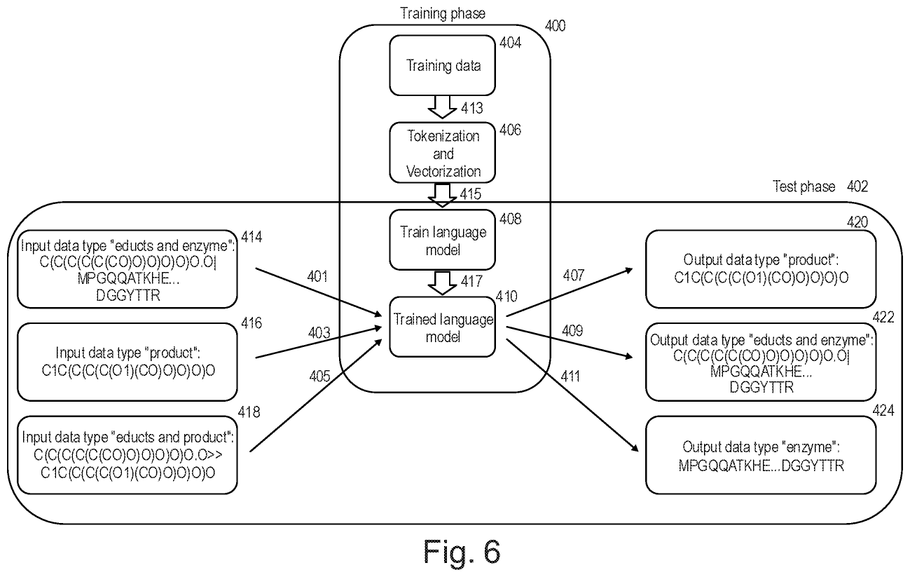

For example, the natural language processing model may be configured to perform a tokenization step on the string-representations provided as input to the model. Thereby, a list of tokens is generated. A token is a fundamental unit that is mapped to a learnable vector that is fed as a sequence element into the machine translation model (e.g., the transformer model—see figure description of FIG. 6).

In an embodiment, a product-prediction model or a precursors-prediction model or an enzyme-prediction model is provided, wherein the model has a sequence-to-sequence model architecture. The sequence-to-sequence model may be a model using artificial recurrent neural networks (e.g. long short-term memory (LSTM), Gated recurrent units (GRUs), etc.).

In yet another embodiment, a product-prediction model or a precursors-prediction model or an enzyme-prediction model is provided, wherein the model has a transformer model architecture. This may be advantageous because transformer models are achieving state-of-the-art results in most tasks. The transformer model may be based on self-attention layers and/or feedforward neural networks.

To be more particular, the token list is converted in list vectors that are inputted into the machine learning models (during training as well as test phase). The mapping between the vectors and the tokens is bijective and guarantees a one-to-one correspondence between a token and a vector. The conversion of the list of tokens into a list of vectors is referred to as the vectorization step.

The tokenization can be performed in multiple ways. In the case of SMILES, the tokenization can comprise identifying atoms and use the characters or groups of characters representing those as tokens. In the case of amino acid sequences, the tokenization can comprise generating tokens which are a mixture of single amino acids and groups of amino acids (multiple characters occurring at a high frequency in a database of protein sequences).

According to some examples, the machine learning model may be a deep learning model. Deep-learning methods are machine learning models that use representation-learning methods with multiple levels of representation, obtained by composing simple but non-linear modules that each transform the representation at one level (starting with the input sequence) into a representation at a higher, slightly more abstract level. Some of these deep learning model may also have transformer architecture and therefore considered to also be transformer models.

According to some examples the reaction of interest is a biochemical reaction.

According to some examples at least one of the one or more educts and/or at least one of the one or more products is an organic molecule selected from a group consisting of a sugar, ethanol, a fatty acid, an ester, an ether, an aliphatic polymer and an aromatic polymer.

According to some examples the computer-implemented method further comprises inputting one or two input strings into the at least one trained machine learning model, each input string being selected from a group of strings consisting of a string representation of one or more educts of the chemical reaction of interest, a string representation of one or more products of the chemical reaction of interest and a string representation of amino acids of an enzyme which was supposed to transform the educts into the products in the reaction of interest. The computer-implemented method further comprises computing by the trained machine learning model a prediction result. The prediction result comprises the one or more strings of the groups of strings which were not provided as input. The prediction is performed as a function of the one or two strings provided as input. The computer-implemented method further comprises outputting the prediction result for predicting or optimizing the chemical reaction of interest.

It is understood that one or more of the aforementioned embodiments of the invention may be combined as long as the combined embodiments are not mutually exclusive.

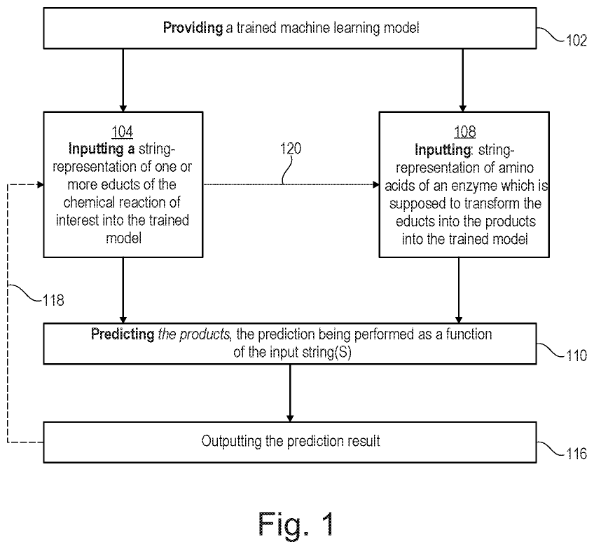

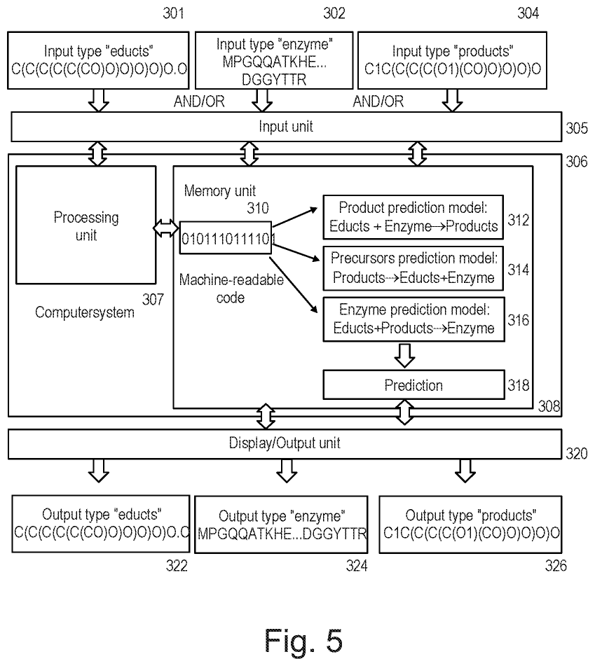

With reference now to FIG. 1, a computer-implemented method is illustrated. The computer-implemented method comprises providing 102 a trained machine learning model. The provided model can be a product prediction model 312 as described, for example, with reference to FIG. 5. The step 102 can comprise, for example, training an untrained version of the one or more models to be provided on training data, or receiving one or more already trained model via a network connection or from a storage device and/or instantiating the one or more trained models. The model provided in step 102 has learned during a training step to correlate a combination of the string representations of atoms and atom groups of one or more educts and of the string representation of an amino acid sequence of an enzyme with a string representation of the atoms and atom groups of one or more products. The model is configured to receive the string representations of the educts and the enzyme as input and to output a predicted string representation of one or more products.

The method further comprises inputting 104 a string representation of one or more educts of the chemical reaction of interest into the trained model. In addition, a string representation of amino acids of an enzyme which is supposed to transform the educts into the products is input 108 into the trained model. Typically, the educts and products are input into the model in a single step.

The trained machine learning model predicts 110 the products 326. The prediction is being performed as a function of the input string(s) provided in 104 and 106.

The computer-implemented method further comprises outputting 116 the prediction result by the model. For example, the outputting can comprise displaying the products or the complete chemical reaction comprising the educts, the enzyme and the predicted one or more products on a display. In some example implementations, the outputting can comprise printing the prediction result. In some further example implementations, the result is sent to a chemical synthesis unit together with a command to automatically perform the predicted chemical reaction. In addition, or alternatively, the prediction result is sent to a software program configured to perform further analysis of the predicted products and/or to a software program configured to automatically order all required educts and the enzyme.

Optionally, the output of a single product-prediction step might be used recursively 118 to generate multi-step synthesis plans. As shown in step 118 the output of the product-prediction model is fed back as a new input into a subsequent product-prediction model, wherein the product of a first product-prediction model then becomes the new educt of a second product-prediction model and the product of a second product-prediction model then becomes the new educt of a third product-prediction model and so on. In addition, or alternatively, the input string of the amino acid sequence of the enzyme is also updated 120 as a result of the multi-step, recursive usage of the product-prediction model. This embodiment of the invention might be advantageous to predict the end product of a multi-step enzymatic cleavage, such as the enzymatic cleavage of complex proteins into amino acids.

For example, the following reaction regulates the chromatin structure in cells:

L-lysyl-[histone]+S-adenosyl-L-methionine=>H(+)+N(6)-methyl-L-lysyl-[histone]+S-adenosyl-L-homocysteine

The following is a SMILES representation of the educts of the aforementioned reaction:

C([C@@H](C(*)═O)N*)CCC[NH3+].C[S+](CC[C@H]([NH3+])C([O—])═O)C[C@H]1 O[C@H]([C@H](O)[C@@H]1O)n1cnc2c(N)ncnc12

The following is a SMILES representation of the products of the aforementioned reaction:

[H+].C([C@@H](N*)CCCC[NH2+]C)(═O)*.Nc1ncnc2n(cnc12)[C@@H]1O[C@H](CSCC[C@H]([NH3+])C([O—])═O)[C@@H](O)[C@H]1O

The enzyme of the aforementioned reaction has the following amino-acid sequence:

MAAPSVPTPLYGHVGRGAFRDVYEPAEDTFLLLDA
LEAAAAELAGVEICLEVGAGSGVVSAFLASMIGPR
ALYMCTDINPEAAACTLETARCNRVHVQPVITDLV
HGLLPRLKGKVDLLVFNPPYVVTPPEEVGSRGIEA
AWAGGRNGREVMDRFFPLAPELLSPRGLFYLVTVK
ENNPEEIFKTMKTRGLQGTTALCRQAGQEALSVLR
FSKS,MAGENFATPFHGHVGRGAFSDVYEPAEDTF
LLLDALEAAAAELAGVEICLEVGSGSGVVSAFLAS
MIGPQALYMCTDINPEAAACTLETARCNKVHIQPV
ITDLVKGLLPRLTEKVDLLVFNPPYVVTPPQEVGS
HGIEAAWAGGRNGREVMDRFFPLVPDLLSPRGLFY
LVTIKENNPEEILKIMKTKGLQGTTALSRQAGQET
LSVLKFTKS.

The tokenization step converts the SMILES representations at the atomic level, for example:

C[S+](CC[C@H]([NH3+])C([O—])═O)C[C@H]1O[C@H]([C@H](O)[C@@H]1O)n1cnc2c(N)ncnc12

“C”,“[S+]”,“(”,“C”,“C”,“[C@H]”,“(”,“[NH3+]”,“)”,“C”,“(”,“[O—]”,“)”,“=”,“O”,“)”,“C”,“[C@H]”,“1”,“O”,“[C@H]”,“(”,“[C@H]”,“(”,“O”,“)”,“[C@@H]”,“1”, “O”,“)”,“n”,“1”,“c”,“n”,“c”,“2”,“c”,“(”,“N”,“)”,“n”,“c”,“n”,“c”,“1”,“2”

The tokenization step converts the amino acid sequence representations at the amino acid group level, for example (considering a part of the sequence for brevity):

MAAPSVPTPLYGHVGRGAFRDVYEPAEDTFLLLDALEAAAAELAGVEICLEV

“MAA”, “PSVP”, “TPL”, “YG”, “HVG”, “RG”, “AF”, “RD”, “VYE”, “P”, “AED”, “TF”, “LLL”, “D”, “ALE”, “AAAA”, “ELAG”, “VE”, “ICL”, “EV”.

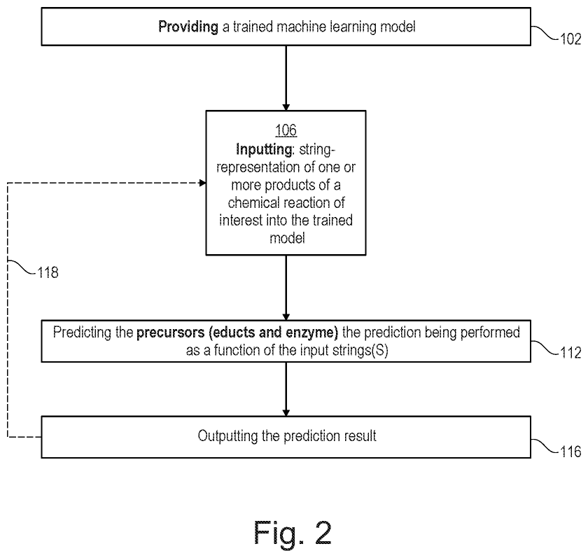

With reference now to FIG. 2, a further variant of a computer-implemented method for predicting aspects of a chemical reaction is illustrated.

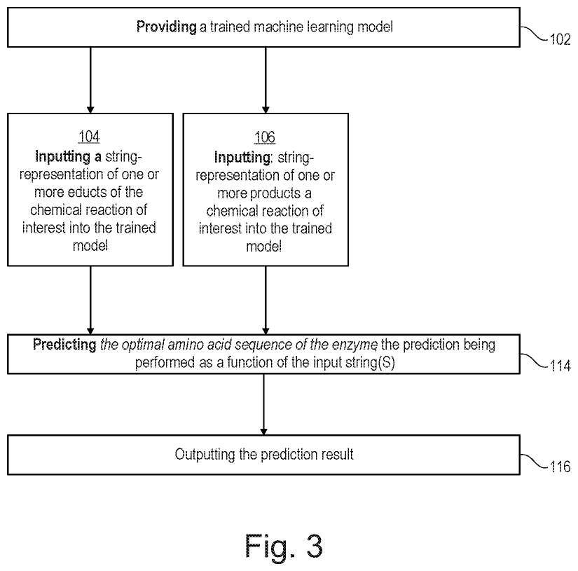

The computer-implemented method comprises providing 102 a trained machine learning model. The model can be provided in the same manner as described with reference to FIG. 1. The provided model is a model having learned in a training step based on a plurality of known chemical reactions comprised in a training data set to correlate one or more products with a combination of one or more educts and an enzyme. An example of such a model is the model 314 described with reference to FIG. 3. The trained machine learning model is configured to predict the precursors (educts and enzyme) based on one or more products provided as input

The computer-implemented method further comprises inputting 106 a string representation of one or more products of a chemical reaction of interest into the trained machine learning model.

The trained machine learning model predicts 112 the precursors (educts and enzyme) based on one or more products provided as input. The prediction is being performed as a function of the input string(s) being string representation of the one or more products.

The computer-implemented method further comprises outputting the prediction result 116. The outputting can be performed, for example, as described with reference to FIG. 1.

Optionally, the output of a single-step precursors-prediction model might be used recursively 118 to generate multi-step synthesis models. As shown in step 118 the output of the precursors-prediction model is fed back as a new input into a subsequent precursors-prediction model, wherein the output educt of a first precursors-prediction model then becomes the new input product of a second precursors-prediction model, and the output educt of a second precursors-prediction model then becomes the new input product of a second precursors-prediction model and so on. This embodiment of the invention might be advantageous to generate a synthesis plan for a given complex product that involves several steps with intermediate products. In addition, this might be advantageous when used for the planning of the retrosynthesis of complex organic molecules.

With reference now to FIG. 3, a further variant of a computer-implemented method for predicting an aspect of a chemical reaction is illustrated. The computer-implemented method comprises providing 102 a trained machine learning model. The provided model can be, for example, the model 316 described with reference to FIG. 3. The provided model is configured to predict the (optimum) amino acid sequence based on the string representations of one or more educts and of one or more products provided as input.

The computer-implemented method further comprises inputting 104 a string representation of one or more educts of the chemical reaction of interest into the trained model. The computer-implemented method further comprises inputting 106 string representations of one or more products of a chemical reaction of interest into the trained model. The trained machine learning model 410 predicts 114 the optimal amino acid sequence of the enzyme. The prediction is performed as a function of the input string(s). The computer-implemented method further comprises outputting 116 the prediction result.

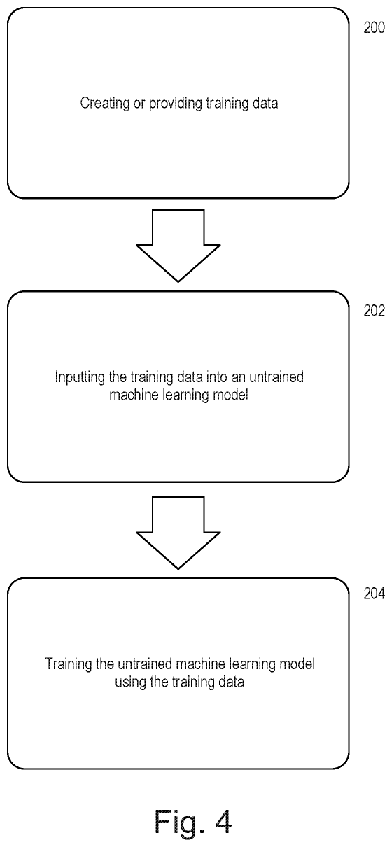

With reference now to FIG. 4, a computer-implemented method for training the untrained machine learning model is illustrated. The method for training the untrained machine learning model comprises creating or providing 200 training data 404, inputting 202 the training data 404 into an untrained machine learning model, training 204 the untrained machine learning model using the training data 404.

For example, the training data set may comprise a large number of known chemical reactions, each comprising one or more educts, one or more products and an enzyme which is adapted to transform the educts into the products. Optionally, each reaction and/or each molecule in the training data comprises metadata, e.g., metadata about environmental parameters of the reaction, e.g., a preferred temperature range, a preferred pH range, the toxicity or commercial availability of the substance, etc. In addition, each educt, each product and each enzyme are comprised in the training data set as a string representation of its atoms and atom groups.

The training data may be input into one or more untrained machine learning models, whereby different models may be trained in a training phase to learn different types of correlation. For example, a product prediction model may learn to correlate the string representations of educts and the enzyme on the one hand and the string representations of the products on the other hand. The precursors prediction model may learn to correlate the string representations of the one or more products on the one hand with string representations of one or more educts and of the enzyme on the other hand. Hence, with the same training data, multiple different trained models may be obtained which may be used in combination to predict chemical reactions, optimal amino acids of enzymes and computationally verify these prediction results.

With reference now to FIG. 5, a computer system 308 for predicting at least one aspect of an enzymatically catalyzed chemical reaction of interest is illustrated. The computer system 308 comprises an input unit 305, a processor unit 307, a memory unit 310 and a display/output unit 302. Different input types can be used as inputs into the input unit 305. Input types comprise input from type “educts” 301, input from type “enzymes” 302, input from type “products” 304. At least one or more of the input types are inputted into the input unit 305. The processing unit 307 of the computer system 308 executes the machine-readable code that is stored in the memory unit 310. The execution of the machine-readable code in the memory unit 310 causes the memory unit 310 to provide various models. The product prediction model 312 describes the reaction of the educts and the enzyme to generate the products. The precursors prediction model 314 describes a reaction of the products to generate educts and the enzyme. The enzyme-prediction model 316 describes the reaction of the educts and the products to generate an enzyme. These models can be used to provide a prediction 318. The prediction can be displayed on the display/output unit 320. The prediction 318 may comprise one of the following output types: output from type “educts” 322, output from type “enzymes” 324, and/or output from type “products” 326. Depending on which input type is provided into the computer system the corresponding output type will be provided by the prediction model. An example workflow comprises the following steps: input from input type “educts” and input type “enzymes”, 301 and 302 respectively is inputted into the input unit 305, the input unit causes the processing unit 307 to execute the machine-readable code 310 that is stored in the memory unit 308, the machine-readable code 310 provides the model from model type 312 “Product prediction model”, wherein educts+enzymes generates a product, the model from model type “Product prediction model” is executed, the model 312 generates a prediction 318, the prediction is displayed on the output unit 320, the output unit 320 displays the output from output type “products” 326.

Another workflow implemented in the computer system 308 might include the following: input from input type “educts” 301 and input from input type “products” 304 are inputted into the input unit 305, the input unit causes the processing unit 307 to execute the machine-readable code 310 that is stored within the memory unit 308, the machine-readable code 310 provides a model from model type “enzyme prediction model” 316, wherein educts+products generate an enzyme, the model from model type “enzyme prediction model” 316 provides a prediction result 318, the prediction result is displayed on the output unit 320, an output from output type “enzyme” 324 is generated, the enzyme is displayed on the display/output unit 320.

In yet another workflow which may be implemented in the machine-readable code 310 input from input type “Products” is inputted into the input unit 305, the input unit causes the processing unit 307 to execute the machine-readable code 310 that is stored within the memory unit 308, the machine-readable code provides a model from model type “Precursors prediction model” 314, wherein products generate educts+enzyme, the model from model type “Precursors prediction model” provides a prediction result 318, the prediction result is displayed on the output unit 320, in this case the outputs from output type “educts” 322 and from output type “enzyme” 324 are provided on the display unit 320.

With reference now to FIG. 6, a computer-implemented method for predicting at least one aspect of an enzymatically catalyzed chemical reaction of interest is illustrated. The computer-implemented method comprises a training phase 400 and a test phase 402. In the training phase 400 training data 404 are provided. The training data 404 are inputted 413 into a machine learning model that can be for example a natural language processing model. The training data 404 are tokenized and vectorized 406. Tokenization 406 involves breaking the raw text into small chunks. A token is a fundamental unit that is mapped to a learnable vector that is fed as a sequence element into the machine learning model. Starting from the string we generate a list of tokens via the so-called tokenization step 406. Such list is then converted in a list vectors that are inputted 415 into the machine learning models 408. The mapping between the vectors and the tokens is bijective and guarantees a one-to-one correspondence between a token and a vector. The conversion between the list of the tokens into the list of vectors is called vectorization 406. The tokenization 406 can be performed in multiple ways. In the case of SMILES, we propose a tokenization 406 based on identifying atoms and use the characters or group of characters representing those as tokens. In the case of amino acid sequences, we propose a learned tokenization that besides single amino acids is based on groups of amino acids (multiple characters occurring with a high frequency in database of protein sequences). The vector is then used as the input into the language model, step 415. The language model is then trained, step 408. The language model may be a machine learning model, a natural language processing model, a sequential data model or a deep learning model. As a result of the training of the language model a trained language model is generated 410.

402 shows the test phase of the prediction model. Input from input data type “educts and enzyme” 414 is inputted 401 into the trained language model 410. The trained language model 410 provides output data from output data type “product” 420. This step, 407, is known as the product prediction model. Input from input data type “product” 416 is inputted 403 into the trained language model 410. The trained language model 410 provides a prediction 40 that generates the output from output data type “educts and enzyme” 422. In the enzyme prediction model input from input data type “products and educts” is inputted 405 into the trained language model 410. The trained language model 410 makes a prediction 411 and provides the output from output data type “enzyme”.

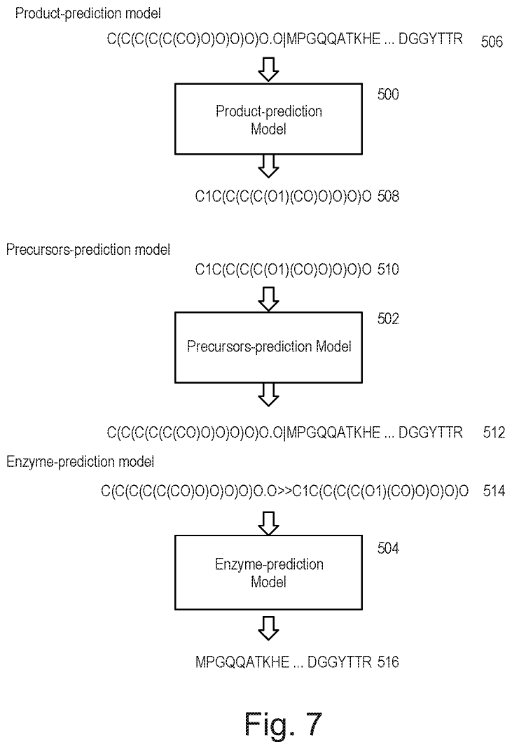

With reference now to FIG. 7, three different computer-implemented models are illustrated. In the product-prediction model one or more educts and an enzyme 506 are inputted into the model 500. The product-prediction model 500 then predicts the product 508. For example, the educts may be inputted into the product prediction model as the SMILES sequence “C(C(C(C(C(CO)O)O)O)O)O.O” and the enzyme may be inputted as the amino acid sequence “MPGQQATKHE . . . DGGYTTR”. The product-prediction model than predicts the product as having the following sequence “C1C(C(C(C(O1)(CO)O)O)O)O”.

In the precursors-prediction model the product 510 is inputted into the model 502. The precursors-prediction model 502 then predicts the educts and the enzyme 512. For example, the product may be inputted into the precursors-prediction model as the SMILES sequence “C1C(C(C(C(O1)(CO)O)O)O)O”. The precursors-prediction model then predicts the educts as having the following SMILES sequence “C(C(C(C(C(CO)O)O)O)O)O.O” and the enzyme as having the following amino acid sequence “MPGQQATKHE . . . DGGYTTR”

In the enzyme-prediction model 504 the products and the educts 514 are inputted into the model 504. The enzyme-prediction model 504 then predicts the enzyme 516. For example, the educts may be inputted in the enzyme-prediction model as having the following SMILES sequence “C(C(C(C(C(CO)O)O)O)O)O.O” and the product may be inputted in the enzyme-prediction model as having the following SMILES sequence “C1C(C(C(C(O1)(CO)O)O)O)O”. The enzyme-prediction model then predicts the enzyme as having the following amino acid sequence “MPGQQATKHE . . . DGGYTTR”.

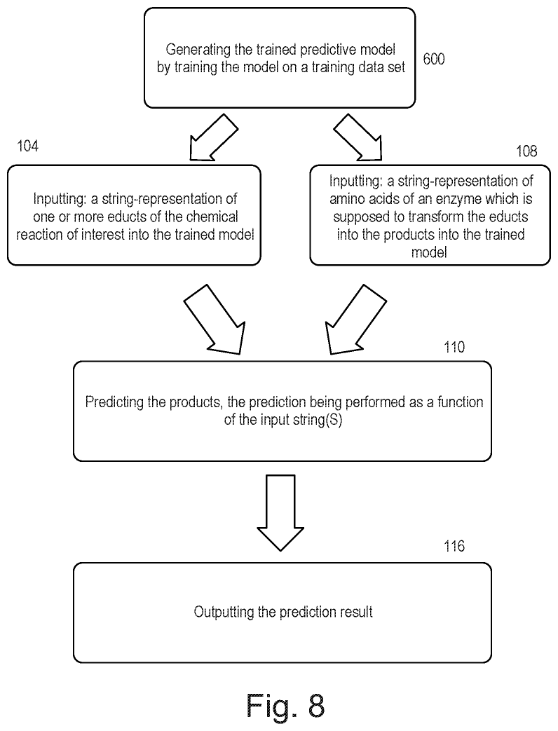

With reference now to FIG. 8, a flowchart of a computer-implemented method for predicting the products of a chemical reaction of interest is illustrated. The computer-implemented method comprises generating 600 the trained predictive model 410 by training the model on the training dataset 404. The computer-implemented method further comprises inputting 104 a string representation of one or more educts of the chemical reaction of interest into the trained model 410 as well as inputting 108 a string representation of amino acids of the enzyme which is supposed to transform the educts into the products into the trained model 410. The trained model 410 then predicts 110 the products. The prediction 110 is being performed as a function of the input string(S). The computer-implemented method further comprises outputting 116 the prediction result.

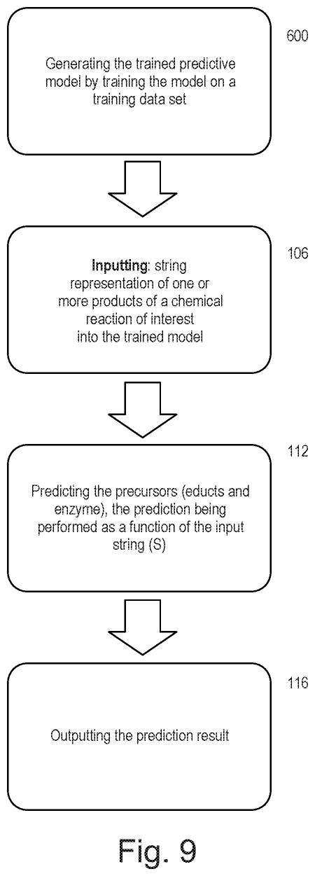

With reference now to FIG. 9, a flowchart for predicting the precursors is illustrated. The computer-implemented method comprises generating 600 the trained predictive model 410 by training the model on a training dataset 404. The computer-implemented method further comprises inputting 106 a string representation of one or more products of a chemical reaction of interest into the trained model 410. The trained model 410 then predicts 112 the precursors (educts and enzymes). The prediction 112 is being performed as a function of the input string(S). The computer-implemented method further comprises outputting 116 the prediction result.

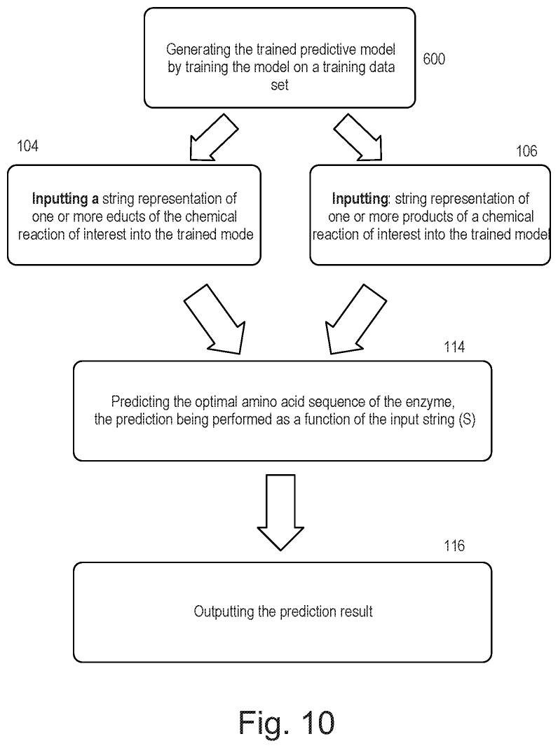

With reference now to FIG. 10, a flowchart is illustrated that shows the prediction of the optimal amino acid sequence of an enzyme. The computer-implemented method comprises generating 600 the trained predictive model 410 by training the model on the training dataset 404. The computer-implemented method further comprises inputting 104 a string representation of one or more educts of the chemical reaction of interest into the trained model 410. The computer-implemented method further comprises inputting 106 a string representation of one or more products of a chemical reaction of interest into the trained model 410. The trained model 410 predicts 114 the optimal amino acid sequence of the enzyme. The prediction 114 is being performed as a function of the input string(S). The computer-implemented method further comprises outputting 116 the prediction result.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application of technical improvement of other technologies found in the marketplace, or to enable others of ordinary skill in the art to understand the embodiments disclosed herein. The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method for predicting at least one aspect of an enzymatically catalyzed chemical reaction of interest, the computer-implemented method comprising:
providing at least one trained machine-learning model, wherein the model was trained to correlate a string-representation of one or more educts, a string-representation of one or more products and a string-representation of amino acids of an enzyme which transforms the educts into the products;
inputting one or two input strings into the at least one trained model, each input string being selected from a group of strings consisting of: a string-representation of one or more educts of the chemical reaction of interest, a string-representation of one or more products of the chemical reaction of interest, and a string-representation of amino acids of an enzyme which is supposed to transform the educts into the products in the reaction of interest;
predicting, by the at least one trained model, the one or more strings of the groups of strings which were not provided as input, the prediction being performed as a function of the one or two strings provided as input; and
outputting the prediction result for predicting or optimizing the chemical reaction of interest.

2. The computer-implemented method of claim 1, further comprising:
generating the trained predictive model by training the model on a training data set, the training data set comprising: a plurality of known enzymatically catalyzed chemical reactions, each reaction in the training data set specifying one or more educts, one or more products, and the enzyme which transforms the educts into the products,
wherein the molecular composition and structure of the educts, the products and the enzyme are specified in a string-representation.

3. The computer-implemented method of claim 1,
wherein the aspect to be predicted is the one or more products that will be generated in the enzyme-catalyzed chemical reaction of interest,
wherein the at least one predictive model comprises a trained product-prediction model which is used for performing the prediction, the product-prediction model being adapted for predicting a string-representation of one or more products of the chemical reaction of interest as a function of the string representation of one or more educts and of the amino acids of the enzyme;
wherein the one or two input strings which are input into the trained model are:
the string-representation of the one or more educts of the chemical reaction of interest and the string-representation of amino acids of the enzyme which is supposed to transform the educts into the products in the reaction of interest; and
wherein the one or more strings predicted by the model is the string-representation of the one or more products.

4. The computer-implemented method of claim 1,
wherein the aspect to be predicted is the precursors required for producing the one or more products, the precursors comprising the educts and the enzyme of the chemical reaction of interest;
wherein the at least one predictive model comprises a trained precursors-prediction model which is used for performing the prediction, the precursors-prediction model being adapted for predicting a string-representation of one or more educts and a string-representation of the amino acid sequence of the enzyme of the chemical reaction of interest as a function of the string representation of one or more products;
wherein the one or two input strings which are input into the trained model is the string-representation of the one or more products of the chemical reaction of interest; and
wherein the one or more strings predicted by the model comprises: the string-representation of the one or more educts and the string-representation of amino acids of the enzyme which is supposed to transform the educts into the products in the reaction of interest.

5. The computer-implemented method of claim 4, further comprising:
using the precursors-prediction model recursively, wherein the output of a previous execution precursors-prediction model is used as the input of a subsequent execution of the precursors-prediction model, wherein the recursive usage of the model generates the multi-step synthesis plan, wherein preferably the synthesis plan is considered to be completed and the recursive use is automatically ended once a terminating condition is met;
wherein the terminating condition is selected from a group consisting of: all predicted educts are commercially available, all predicted educts are non-toxic, all predicted educts are water-soluble, and the predicted educts meet a predefined requirement.

6. The computer-implemented method of claim 1,
wherein the aspect to be predicted is the optimal amino acid sequence of an enzyme capable of catalyzing the chemical reaction of interest,
wherein the at least one predictive model comprises: a trained enzyme-prediction model which is used for performing the prediction, the enzyme prediction model being adapted for predicting a string-representation of the amino acid sequence of an enzyme optimally capable of catalyzing the chemical reaction of interest as a function of the string representation of the one or more educts, and the one or more products of the chemical reaction of interest;
wherein the one or two input strings which are input into the trained model are: the string-representation of the one or more educts, and the string-representation of the one or more products; and
wherein the one or more strings predicted by the model is the string-representation of amino acids of the enzyme which is supposed to be the optimum amino acid sequence for an enzyme capable of transforming the educts into the products in the reaction of interest.

7. The computer-implemented method of claim 6, further comprising:
inputting the string-representation of the predicted optimum amino acid sequence and the string-representation of the one or more educts of the reaction of interest into the at least one predictive model;
in response to the inputting, predicting, by the trained model, a string-representation of one or more products to be generated by an enzyme having the predicted optimum amino acid sequence from the one or more educts based on the inputted string-representations;
determining if the said one or more predicted products are identical to the one or more products used as input for predicting the optimum amino acid sequence of the enzyme;
if the products are identical, considering the predicted optimum amino acid sequence as verified; and
if the products are not identical, considering the predicted optimum amino acid as non-verified and unreliable.

8. The computer-implemented method of claim 1,
wherein in addition to the input strings, additional information is input into the at least one trained model wherein the additional information is one or more of the following: toxicity information of at least some of the educts and/or products, efficiency information of the chemical reaction of interest, solubility information of at least some of the educts and/or products and/or the enzymes, and selectivity information of the chemical reaction of interest; and
wherein the trained predictive model is configured to perform the prediction such that at least one of the following is optimized: the toxicity of the educts and/or products of the chemical reaction of interest is minimized, the quantities of necessary solvents are minimized, the need for organic solvents is minimized, the amount of unwanted side products is minimized, the yield of a target product is maximized.

9. The computer-implemented method of claim 1, further comprising:
creating a line notation being indicative of the structure and molecular composition of each of the educts using a sequence of string elements, wherein each string element is in particular one of the following: a unicode character, an artificially created character representing an atom or atom group, and a group of adjacent characters together representing an atom or atom group,
wherein the line notation is in particular the simplified molecular-input line-entry system (SMILES), the Wiswesser line notation (WLN), ROSDAL, SYBYL Line Notation (SLN) or SMILES arbitrary target specification (SMARTS) or a one-letter or three-letter amino acid sequence in case the educt or product is a peptide or a protein;
if the one or more educts comprise more than one educt, concatenating the line notations of each of the one or more educts and one or more delimiters to obtain a concatenate to be used as the line notation of the educts; and
using the line notation as the string-representation of the one or more educts.

10. The computer-implemented method of claim 9, wherein creating the line notation of the one or more educts comprises:
analyzing the molecular composition of a plurality of known molecules for identifying a predefined number of chemical groups occurring most frequently in the analyzed molecules, each chemical group comprising a plurality of atoms;
representing each of the atoms of the educts not being member of one of the identified chemical groups by a respective, atom-specific symbol;
identifying chemical groups by a single symbol; and
representing each of the identified chemical groups occurring in the one or more educts by a respective, chemical-group-specific symbol, the chemical-group-specific symbols being different from the atom-specific symbols.

11. The computer-implemented method of claim 1, further comprising:
creating a line notation of amino acids of the enzyme, wherein the line notation covers all amino acids or covers at least the amino acids of the enzymatically active moiety of the enzyme, wherein the line notation is in particular the one-letter or three-letter amino acid code sequence; and
using the line notation as the string-representation of the enzyme.

12. The computer-implemented method of claim 11, wherein creating the line notation of the one or more products comprises:
analyzing the amino acid sequences of a plurality of known enzymes for identifying a predefined number of amino acid sub-sequences occurring most frequently in the analyzed enzymes, each amino acid sub-sequence comprising two or more amino acids;
representing each of the amino acids of the enzyme not being member of one of the identified sub-sequences by a respective, amino-acid-specific symbol; and
representing each of the identified amino acid sub-sequences occurring in the enzyme by a respective, sub-sequence-specific symbol, the sub-sequence-specific symbols being different from the amino-acid-specific symbols.

13. The computer-implemented method of claim 1,
wherein the at least one machine learning model is or comprises a model selected from a group consisting of: a natural language processing (NLP) model adapted to translate strings representing sentences in a source language into strings representing sentences in a target language and a machine translation model, wherein the machine translation model has neural machine translation architecture, a non-supervised machine-learning model, a supervised machine-learning model, a semi-supervised machine-learning model.

14. The computer-implemented method of claim 1, wherein the at least one machine learning model is a natural language processing model selected from a group consisting of: a sequence-to-sequence model and a transformer model.

15. A computer program product for predicting at least one aspect of an enzymatically catalyzed chemical reaction of interest, the computer program product comprising a computer readable storage medium having program instructions embodied therewith, the program instructions being executable by a processing circuit and cause the processing circuit to:
providing at least one trained machine-learning model, wherein the model was trained to correlate a string-representation of one or more educts, a string-representation of one or more products and a string-representation of amino acids of an enzyme which transforms the educts into the products;
inputting one or two input strings into the trained model, each input string being selected from a group of strings consisting of: a string-representation of one or more educts of the chemical reaction of interest, a string-representation of one or more products of the chemical reaction of interest, and a string-representation of amino acids of an enzyme which is supposed to transform the educts into the products in the reaction of interest;
predicting, by the at least one trained model, the one or more strings of the groups of strings which were not provided as input, the prediction being performed as a function of the one or two strings provided as input; and
outputting the prediction result for predicting or optimizing the chemical reaction of interest.

16. A computer system for predicting at least one aspect of an enzymatically catalyzed chemical reaction of interest, the computer system comprising a processor and a computer readable medium, wherein the computer-readable medium comprises:
at least one trained machine-learning model, wherein the model was trained to correlate a string-representation of one or more educts, a string-representation of one or more products and a string-representation of amino acids of an enzyme which transforms the educts into the products;
computer-readable program code that causes the processor to:
input one or two input strings into the trained model, each input string being selected from a group of strings consisting of: a string-representation of one or more educts of the chemical reaction of interest, a string-representation of one or more products of the chemical reaction of interest, and a string-representation of amino acids of an enzyme which is supposed to transform the educts into the products in the reaction of interest;
predict by the at least one trained model, the one or more strings of the groups of strings which were not provided as input, the prediction being performed as a function of the one or two strings provided as input; and
output the prediction result for predicting or optimizing the chemical reaction of interest.
